(19) United States
(12) Patent Application Publication
(10) Pub. No.: US 2026/0102571 A1
(43) Pub. Date: Apr. 16, 2026

(54) NEBULIZING DEVICE

(71) Applicant: HCMed Innovations Co., LTD., Taipei City (TW)

(72) Inventors: HUNG-WEN CHEN, New Taipei City (TW); CHUN-CHIA JUAN, New Taipei City (TW)

(21) Appl. No.: 19/310,938

(22) Filed: Aug. 27, 2025

Related U.S. Application Data
(60) Provisional application No. 63/690,338, filed on Sep. 4, 2024.

(30) Foreign Application Priority Data
Jul. 15, 2025 (CN) 202510972294.2

(51) Int. Cl. A61M 11/00

(52) U.S. Cl. CPC A61M 11/005; A61M 2205/3327; A61M 2205/3382; A61M 2205/3386; A61M 2205/50

(57) ABSTRACT

A nebulizing device includes a cup body, a nebulizing module, and a detecting module. The cup body includes an opening and a through hole. The opening and the through hole are provided on a top and a bottom of the cup body, respectively. The cup body has a reservoir that is configured to contain a liquid medication, and the reservoir is in fluid communication with the opening and the through hole. The nebulizing module is disposed in the cup body and located above the through hole. The detecting module is disposed in the cup body and located above the nebulizing module. The detecting module has a detection area. An orthogonal projection of the detection area onto a reference plane is completely located within an orthogonal projection of the through hole onto the same reference plane.

### CROSS-REFERENCE TO RELATED PATENT APPLICATION

This application claims the benefit of priority to the U.S. Provisional Patent Application Ser. No. 63/690,338 filed on Sep. 4, 2024, which application is incorporated herein by reference in its entirety.

This application claims the benefit of priority to China Patent Application No. 202510972294.2, filed on Jul. 15, 2025, in the People's Republic of China. The entire content of the above identified application is incorporated herein by reference.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure relates to a nebulizing device, and more particularly to a nebulizing device that is configured to detect residual medication.

### BACKGROUND OF THE DISCLOSURE

An existing nebulizing device typically includes a detection element disposed inside a medication cup to detect the residual amount of liquid medication therein. However, in the related art, the detection element is offset to one side (e.g., the right side) of the medication cup. As a result, when the medication cup is tilted (e.g., tilted to the left side), the residual liquid medication may be located away from the detection element and cannot be effectively detected, thereby leading to inaccurate determination of the residual amount of liquid medication and reducing the precision and reliability of medication usage.

Therefore, how to overcome the above-mentioned problem through an improvement in structural design has become an important issue to be addressed in the related art.

### SUMMARY OF THE DISCLOSURE

In response to the above-referenced technical inadequacy, the present disclosure provides a nebulizing device, so as to address an issue of inaccurate detection of residual medication in the medication cup due to the detection element in existing nebulizing devices often being offset to one side of the cup.

In order to solve the above-mentioned problems, one of the technical aspects adopted by the present disclosure is to provide a nebulizing device, which includes a cup body, a nebulizing module, and a detecting module. The cup body includes an opening and a through hole. The opening and the through hole are provided on a top and a bottom of the cup body, respectively. The cup body has a reservoir that is configured to contain a liquid medication, and the reservoir is in fluid communication with the opening and the through hole. The nebulizing module is disposed in the cup body and located above the through hole. The detecting module is disposed in the cup body and located above the nebulizing module. The detecting module has a detection area. An orthogonal projection of the detection area onto a reference plane is completely located within an orthogonal projection of the through hole onto the same reference plane.

Therefore, in the nebulizing device provided by the present disclosure, by ensuring that the orthogonal projection of the detection area onto a reference plane is completely located within the orthogonal projection of the through hole onto the same reference plane, the detection area can be centrally positioned within the reservoir rather than being offset to one side. Through the structural design of coaxial alignment between the detection area and the through hole, the sensing accuracy of the detecting module for the liquid level in the reservoir can be improved, thereby ensuring consistency in the amount of residual medication, avoiding misjudgment or signal distortion caused by positional deviation of the detecting module, and enhancing the operational stability and safety of the nebulization process.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 is a schematic view of a nebulizing device according to the present disclosure;

FIG. 2 is a schematic exploded view of the nebulizing device according to the present disclosure;

FIG. 3 is a schematic view of a cup body according to the present disclosure;

FIG. 4 is a schematic cross-sectional view of the cup body according to the present disclosure;

FIG. 5 is a schematic view of orthogonal projections of a detection area and a through hole according to the present disclosure;

FIG. 6 is a functional block diagram of the nebulizing device according to the present disclosure; and

FIG. 7 is a schematic cross-sectional view of another implementation of the cup body according to the present disclosure.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a,” “an” and “the” includes plural reference, and the meaning of “in” includes “in” and “on.” Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first,” “second” or “third” can be used to describe various components, signals or the like, which are for distinguishing one component/signal from another one only, and are not intended to, nor should be construed to impose any substantive limitations on the components, signals or the like.

### Embodiment

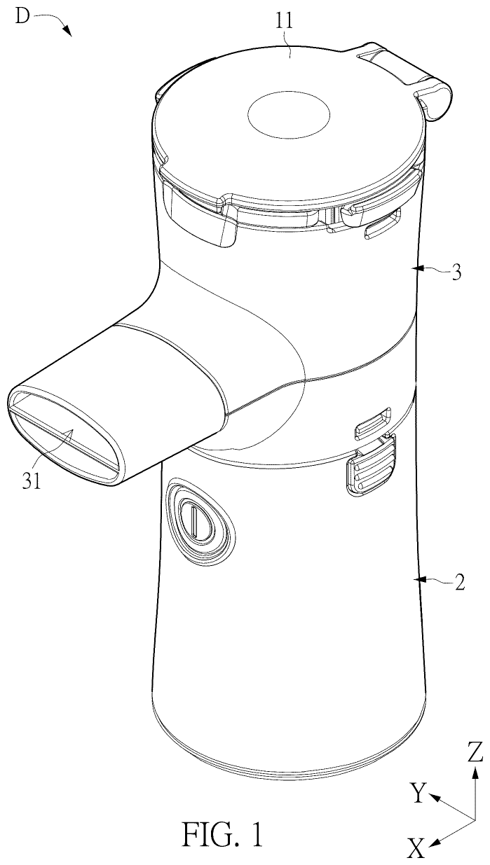

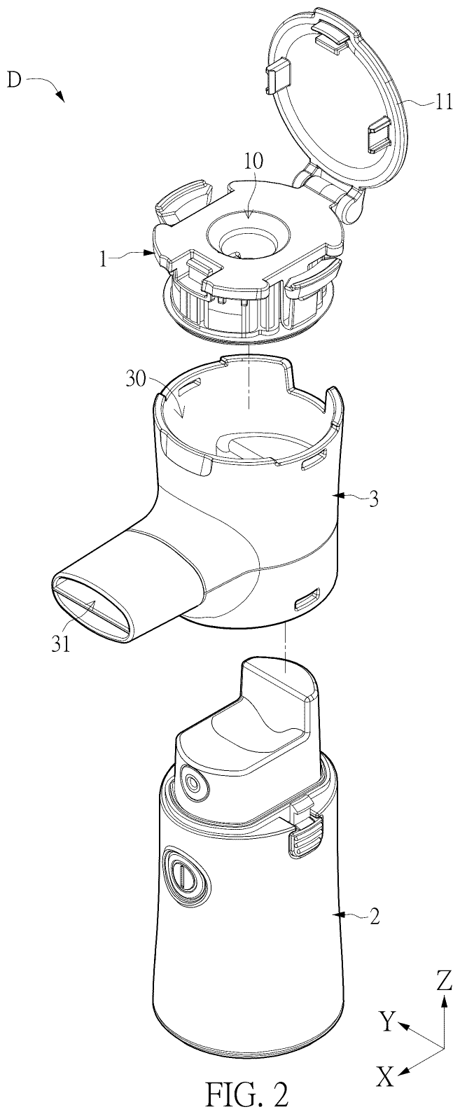

Reference is made to FIGS. 1 and 2. FIG. 1 is a schematic view of a nebulizing device according to the present disclosure. FIG. 2 is a schematic exploded view of the nebulizing device according to the present disclosure. The present disclosure provides a nebulizing device D, which includes a cup body 1, a nebulizer host 2, a housing 3, a nebulizing module 4, and a detecting module 5.

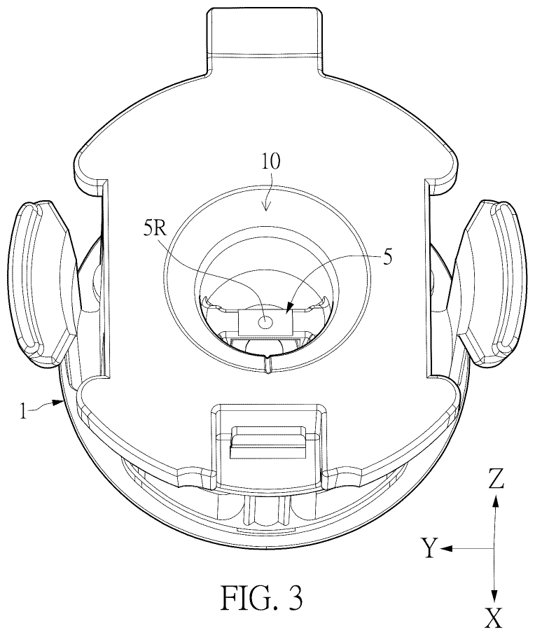

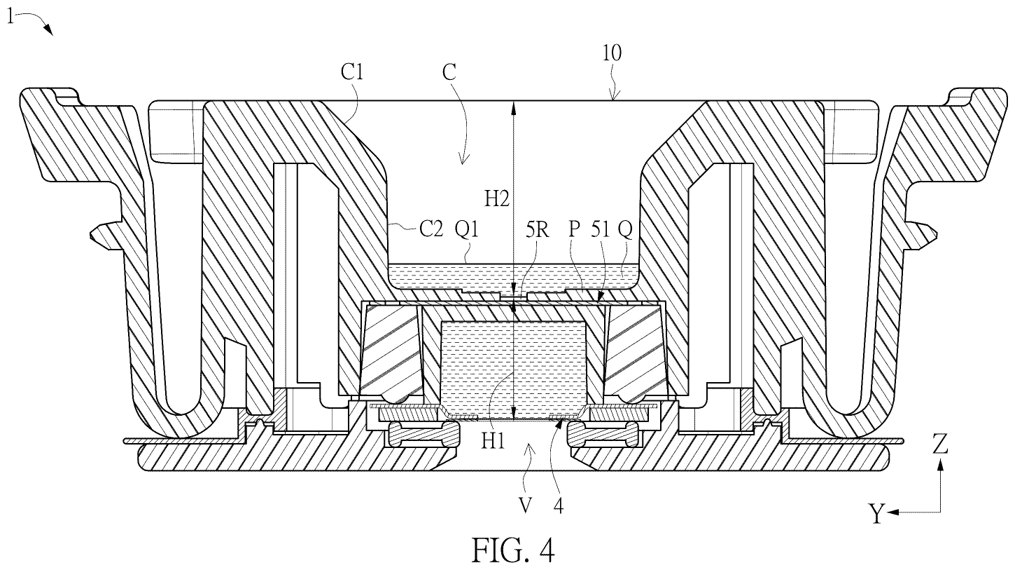

Reference is made to FIGS. 2 to 4. FIG. 3 is a schematic view of a cup body according to the present disclosure. FIG. 4 is a schematic cross-sectional view of the cup body according to the present disclosure. The housing 3 is configured to connect the nebulizer host 2 and the cup body 1. The housing 3 includes an air chamber 30 therein, and the housing 3 includes a mouthpiece 31 that is configured to be in fluid communication with the air chamber 30. The cup body 1 includes an opening 10 and a through hole V. The opening 10 and the through hole V are respectively provided on a top and a bottom of the cup body 1. The cup body 1 has a reservoir C therein, and the reservoir C is configured to contain a liquid medication Q (shown in FIG. 4). The reservoir C is in fluid communication with the opening 10 and the through hole V. Furthermore, the reservoir C is in fluid communication with the air chamber 30 through the through hole V. As shown in FIG. 2, the cup body 1 further includes a lid 11. The lid 11 is pivotally connected to a side of the cup body 1, and is configured to rotatably cover the opening 10. It should be noted that the depiction of the lid 11 is omitted in FIG. 3 for ease of illustration.

As shown in FIG. 4, the reservoir C includes a first inner wall C1 and a second inner wall C2. The first inner wall C1 is located between the opening 10 and the second inner wall C2, and the second inner wall C2 is located between the first inner wall C1 and the through hole V. The first inner wall C1 is funnel-shaped, and an inner diameter of the first inner wall C1 gradually decreases from the opening 10 toward the second inner wall C2, so as to guide the liquid medication Q to flow downward toward the through hole V. The second inner wall C2 is generally cylindrical and has a substantially constant inner diameter, thereby stabilizing the flow rate and direction of the liquid medication Q passing through the through hole V to improve nebulization efficiency.

The nebulizing module 4 and the detecting module 5 are disposed within the cup body 1. The nebulizing module 4 is located directly above the through hole V, while the detecting module 5 is further disposed above the nebulizing module 4. As shown in FIG. 4, the reservoir C is filled with the liquid medication Q.

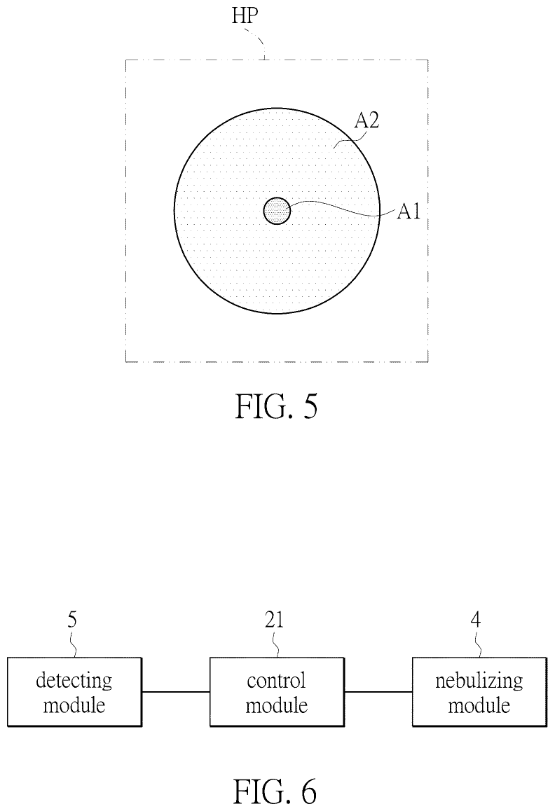

Reference is made to FIGS. 3, 4, and 5. FIG. 5 is a schematic view of orthogonal projections of a detection area and a through hole according to the present disclosure. The detecting module 5 includes a detection area 5R, which is positioned above the through hole V. In other words, when the detection area 5R and the through hole V are orthogonally projected along a vertical direction (i.e., a Z-axis) onto a reference plane, which is an imaginary horizontal projection plane HP in FIG. 5, an orthogonal projection A1 of the detection area 5R and an orthogonal projection A2 of the through hole V are obtained.

In the embodiment of the present disclosure, the area of the orthogonal projection A1 is smaller than that of the orthogonal projection A2, and the orthogonal projection A1 is entirely located within the range of the orthogonal projection A2. That is, the orthogonal projection A1 of the detection area 5R does not exceed the boundary of the orthogonal projection A2 of the through hole V. Preferably, the orthogonal projection A1 is centrally located within the orthogonal projection A2, which means that the detection area 5R and the through hole V are coaxially aligned. The coaxial alignment improves the sensing accuracy of the detecting module 5 in detecting liquid level variations in the reservoir C. This configuration enables the detection area 5R to accurately sense the amount of liquid medication above the through hole V, thereby preventing misjudgment or signal distortion and ensuring stable and safe nebulization.

As shown in FIGS. 3 and 4, the nebulizing module 4 can be composed of a nebulizing plate and a piezoelectric material. The nebulizing module 4 operates based on ultrasonic vibration to drive the nebulizing plate, thereby converting the liquid medication Q in the reservoir C into aerosol particles for inhalation. On the other hand, the detecting module 5 is a liquid level sensor designed to detect the level of the liquid medication Q, so as to control the start, stop, or refill alert of the nebulization process, thereby managing the residual amount of the liquid medication Q. For example, the detecting module 5 can use capacitive or resistive liquid level detection, but the present disclosure is not limited thereto.

In the embodiment of the present disclosure, the detecting module 5 includes a metal piece 51 and a sensing circuit (not shown in the figures) that is electrically connected to the metal piece 51. For instance, the sensing circuit includes a microcontroller and a signal processing unit, but the present disclosure is not limited thereto. The metal piece 51 extends laterally across an upper side of the through hole V, and both ends of the metal piece 51 are stably fixed to the second inner wall C2. Furthermore, the metal piece 51 is covered with an insulating material P, and only a small portion is exposed to form a contact point with the liquid medication Q, which defines the detection area 5R.

In addition, the detection area 5R is located directly above the through hole V, and a first distance H1 between the detection area 5R and the through hole V is less than a second distance H2 between the detection area 5R and the opening 10. When the liquid medication Q is poured into the reservoir C of the cup body 1 and a liquid level Q1 of the liquid medication Q reaches a certain height, the detection area 5R comes into contact with the liquid medication Q, causing the sensing circuit to generate a change in resistance (or capacitance). By detecting this change, a control circuit inside the nebulizer host 2 determines a status of the liquid level Q1 of the liquid medication Q. When the control circuit determines that the liquid level Q1 meets the activation condition, it drives the nebulizing module 4 to initiate nebulization. Conversely, when the liquid level Q1 is lower than the detection area 5R, the sensing circuit stops activating the nebulizing module 4, and may further issue a liquid shortage alert to remind the user to refill.

In the present disclosure, by adjusting the first distance H1 between the detection area 5R and the through hole V, the residual amount of the liquid medication Q in the reservoir C can be controlled, especially for the residual trace amount of the liquid medication Q. When a fixed amount of the liquid medication Q is poured into the reservoir C, and the residual amount after nebulization is also consistent, the nebulized amount of the liquid medication Q can be calculated. Then, this nebulized amount can be used to estimate the dosage inhaled by the user.

Reference is made to FIG. 6, which is a functional block diagram of the nebulizing device according to the present disclosure. The nebulizer host 2 includes a control module 21 (i.e., the above control circuit) that is electrically connected to the nebulizing module 4 and the detecting module 5. The control module 21 is configured to control liquid level detection and nebulization operation.

As shown in FIGS. 4 and 6, when the liquid level Q1 of the liquid medication Q rises above the position of the detection area 5R, the detection area 5R comes into contact with the liquid medication Q. At this time, the detecting module 5 detects that the liquid level has reached the threshold and outputs a detection signal to the control module 21. Based on this signal, the control module 21 determines that the liquid medication Q is sufficient and activates the nebulizing module 4 to nebulize the liquid medication Q. Then, referring to FIG. 2, the liquid medication Q is nebulized and converted into fine aerosol, which diffuses into the air chamber 30 through the through hole V. Finally, when the user inhales through the mouthpiece 31, the aerosol is guided from the air chamber 30 to the mouthpiece 31 and inhaled into the body, thereby completing the delivery process of the liquid medication Q.

On the other hand, when the liquid level Q1 of the liquid medication Q is lower than the position of the detection area 5R (not shown in the figures), the detection area 5R no longer contacts the liquid medication Q. In this case, the detecting module 5 stops outputting a detection signal to the control module 21. Thereby, the control module 21 determines that the residual amount of medication Q has reached the expected level and stops controlling the nebulizing module 4, thereby preventing the nebulizing module 4 from continuing to operate in a liquid shortage state.

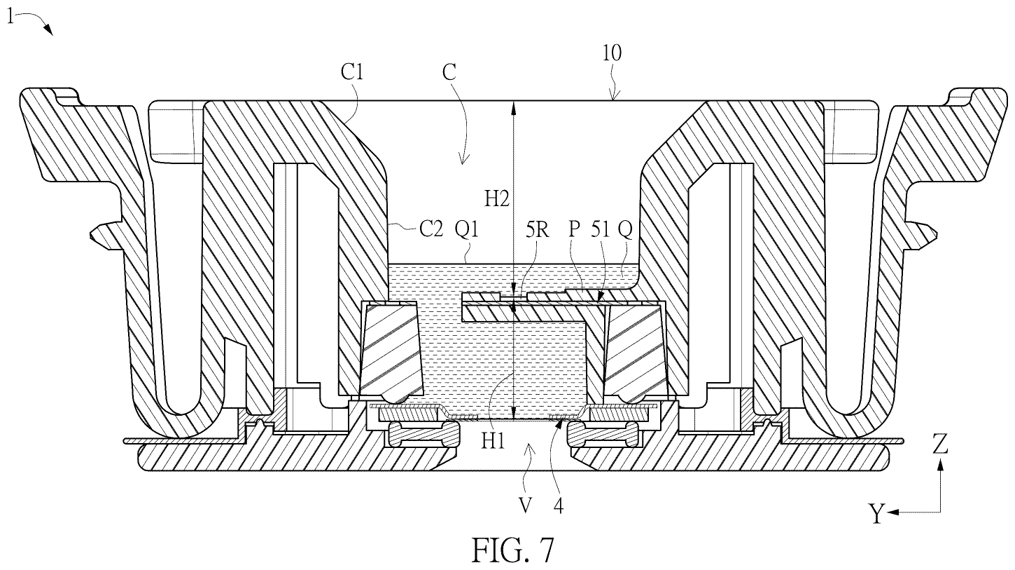

As shown in FIG. 7, in another embodiment of the present disclosure, the metal piece 51 of the detecting module 5 can also extend only from one side of the inner wall of the cup body 1 to a position above the through hole V. Similarly, the metal piece 51 is covered with the insulating material P, leaving a portion exposed to form the detection area 5R. The detection method for the detection area 5R is the same as that described in the previous embodiment and will not be repeated herein.

By arranging the detection area 5R of the detecting module 5 directly above the through hole V, the orthogonal projection A1 of the detection area 5R is entirely located within the orthogonal projection A2 of the through hole V, and preferably at the center of the orthogonal projection A2. This coaxial arrangement ensures that the detection area 5R aligns with the primary flow region of the liquid medication Q entering the through hole V. Through this configuration, even if the cup body 1 is tilted when being held or put down, the detection area 5R can still correspond to the region directly above the through hole V regardless of the tilt direction or angle, thereby accurately detecting the residual medication Q in that area.

Furthermore, even if the user operates the nebulizing device D in different postures and causes the cup body 1 to tilt in various directions, the detecting module 5 can still accurately determine the residual amount of the liquid medication Q above the through hole V when it reaches the preset residual level, thereby ensuring consistency in the detected amount. In other words, when the liquid level Q1 is lower than the detection area 5R and the sensing circuit stops activating the nebulizing module 4, the nebulized amount of the liquid medication Q can be calculated by subtracting the consistent residual amount from the amount of the liquid medication Q initially poured into the reservoir C. This alignment design of positioning the detection area 5R above the through hole V effectively enhances the reliability and consistency of liquid level detection, preventing detection errors or misjudgment of liquid shortage due to tilt, thereby ensuring accurate control of the nebulization process.

### Beneficial Effects of the Embodiments

In the nebulizing device provided by the present disclosure, by arranging the detection area 5R of the detecting module 5 directly above the through hole V, the orthogonal projection A1 of the detection area 5R is entirely located within the orthogonal projection A2 of the through hole V, and preferably at the center of the orthogonal projection A2. This coaxial arrangement ensures that the detection area 5R aligns with the primary flow region of the liquid medication Q entering the through hole V. Through this configuration, even if the cup body 1 is tilted when being held or put down, the detection area 5R can still correspond to the region directly above the through hole V regardless of the tilt direction or angle, thereby accurately detecting the residual liquid medication Q in that area.

Furthermore, since the detection area 5R is focused on detecting the residual liquid medication above the through hole V, the utilization efficiency of the liquid medication can be improved, and a situation where the system prematurely determines a shortage of the liquid medication Q caused by improper positioning of the detection area can be avoided, thereby reducing waste of medication, extending the effective usage time after each refill, and achieving the effects of conserving the liquid medication Q and enhancing the efficiency of the nebulizing device D.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A nebulizing device, comprising:
a cup body including an opening and a through hole, wherein the opening and the through hole are respectively provided on a top and a bottom of the cup body, the cup body has a reservoir therein, the reservoir is configured to contain a liquid medication, and the reservoir is in fluid communication with the opening and the through hole;
a nebulizing module disposed in the cup body and located above the through hole; and
a detecting module disposed in the cup body and located above the nebulizing module, wherein the detecting module has a detection area, and an orthogonal projection of the detection area onto a reference plane is completely located within an orthogonal projection of the through hole onto the same reference plane.

2. The nebulizing device according to claim 1, wherein the detecting module includes a metal piece extending across an upper side of the through hole, the metal piece is covered with an insulating material, and a portion of the metal piece is exposed from the insulating material to define the detection area.

3. The nebulizing device according to claim 1, wherein the detecting module includes a metal piece extending from one side of an inner wall of the cup body to a position above the through hole, the metal piece is covered with an insulating material, and a portion of the metal piece is exposed from the insulating material to define the detection area.

4. The nebulizing device according to claim 1, wherein reservoir includes a first inner wall and a second inner wall, the first inner wall is disposed between the opening and the second inner wall, the second inner wall is disposed between the first inner wall and the through hole, and an inner diameter of the reservoir at the first inner wall gradually decreases from the opening toward the second inner wall.

5. The nebulizing device according to claim 1, wherein a first distance between the detection area and the through hole is less than a second distance between the detection area and the opening.

6. The nebulizing device according to claim 1, further comprising a lid that is pivotally connected to a side of the cup body, wherein the lid is configured to cover the opening.

7. The nebulizing device according to claim 1, wherein the orthogonal projection of the detection area is centrally located within the orthogonal projection of the through hole.

8. The nebulizing device according to claim 1, further comprising a nebulizer host and a housing, the housing is configured to connect the nebulizer host and the cup body, the housing includes an air chamber therein, the housing includes a mouthpiece that is configured to be in fluid communication with the air chamber, and the reservoir is in fluid communication with the air chamber through the through hole.

9. The nebulizing device according to claim 8, wherein the nebulizer host includes a control module that is electrically connected to the nebulizing module and the detecting module; wherein, when a liquid level of the medication is higher than a position of the detection area, the detection area comes into contact with the medication and outputs a detection signal to the control module, such that the control module controls the nebulizing module to nebulize the medication.

10. The nebulizing device according to claim 8, wherein the nebulizer host includes a control module that is electrically connected to the nebulizing module and the detecting module; wherein, when a liquid level of the medication is lower than a position of the detection area, the detection area loses contact with the medication and does not output a detection signal to the control module, such that the control module stops controlling the nebulizing module to nebulize the medication.
